(19) United States
(12) Patent Application Publication
(10) Pub. No.: US 2009/0054991 A1
(43) Pub. Date: Feb. 26, 2009

(54) TRANSFORAMINAL LUMBAR INTERBODY FUSION CAGE

(76) Inventors: Ashok Biyani, Sylvania, OH (US); Geoffrey W. Combs, Canal Winchester, OH (US); Cliff Ryan Walters, Westerville, OH (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 12/258,671

(22) Filed: Oct. 27, 2008

Related U.S. Application Data
(63) Continuation-in-part of application No. 11/742,873, filed on May 1, 2007.
(60) Provisional application No. 60/796,691, filed on May 2, 2006.

(51) Int. Cl. A61F 2/44

(52) U.S. Cl. 623/17.16; 623/17.11

(57) ABSTRACT

A spinal cage system for inserting a spinal cage assembly into a spine to separate and support adjacent spinal vertebrae, includes a first cage member; a second cage member; and an articulating mechanism adapted to connect the first cage member to the second cage member and to permit the first and second cage members to move relate to each other. An insertion instrument is adapted to capture the spinal cage assembly for insertion of the spinal cage assembly into a spine and to rotate the first and second cage members relative to each other to achieve a desired orientation in the spine.

### CROSS-REFERENCE TO RELATED APPLICATIONS

The present application is a continuation-in-part of application Ser. No. 11/742,873, filed on May 1, 2007, which claims the filing benefit of U.S. Provisional Application Ser. No. 60/796,691, filed May 2, 2006, the disclosures of which are hereby incorporated herein by reference in their entirety.

### STATEMENT REGARDING FEDERALLY SPONSORED RESEARCH

Not applicable.

### BACKGROUND

The present disclosure relates generally to the field of orthopedic surgery and, more particularly, to the field of spinal implants.

Fusion cages generally have been used in orthopedic surgery for fixing bones in a pre-selected spacial orientation. However, in inserting such fusion cages using minimally invasive surgical techniques, it is oftentimes difficult to insert a fusion cage without making an incision that is larger than desired or significantly displacing the neural element. Typically, interbody fusion cages of the prior art require considerable space to be rotated into the proper position between adjacent vertebrae. To properly position such prior art cages, it generally was necessary to make a larger incision or displace the nerve roots more than desirable, or both, to properly position the fusion cage.

### BRIEF SUMMARY

The present disclosure overcomes the foregoing and other shortcomings and drawbacks of the interbody fusion cages heretofore known. While the new fusion cage design and insertion method will be described in connection with certain embodiments, it will be understood that the disclosure is not limited to these embodiments. On the contrary, the disclosure includes all alternatives, modifications and equivalents as may be included within the spirit and scope of the present disclosure.

The present disclosure relates to a fusion cage that is used to separate and support adjacent vertebrae in the spine. The fusion cage may be designed for use in the lumbar region of the spine, although it is possible to use the fusion cage of the present disclosure in other areas of the spine as well. The fusion cage has a first spacer member or chamber and a second spacer member or chamber that are pivotally interconnected by an articulating mechanism such as a hinge. The first and second spacer members are designed for insertion between adjacent vertebrae to properly support and separate the vertebrae. An advancing mechanism is located between the first and second spacer members to pivotally move the first spacer member relative to the second spacer member around the hinge. The angular position of the first spacer member relative to the second spacer member facilitates the insertion of the fusion cage around the dural sac and reduces the space necessary for the insertion of the cage. The advancing mechanism is operable to adjust the angular position of the first and second spacer members so that the first and second spacer members are in the desired position relative to the adjacent vertebrae when the cage is fully inserted.

In another embodiment, the spinal cage system includes a first cage member; a second cage member; and an articulating mechanism adapted to connect the first cage member to the second cage member and to permit the first and second cage members to move relate to each other. An insertion instrument is adapted to capture the spinal cage assembly for insertion of the spinal cage assembly into a spine and to rotate the first and second cage members relative to each other to achieve a desired orientation in the spine.

One advantage of the present fusion cage design is the use of an articulated fusion cage that can be displaced during the insertion process to move around the neural element in a manner that takes less room. Such articulation has the advantage of facilitating insertion of the cage during minimally invasive spinal surgery and reducing the need to displace the spinal cord more than is desirable. Another advantage is that, as the present fusion cage is maneuvered into position, the angular relationship between the two portions of the cage can be adjusted so that the cage is in the proper orientation when finally inserted. These and other advantages will be readily apparent to those skilled in the art based on the disclosure set forth herein.

### BRIEF DESCRIPTION OF THE DRAWINGS

For a fuller understanding of the nature and advantages of the present device, system, and insertion method, reference should be had to the following detailed description taken in connection with the accompanying drawings, in which:

FIG. 1 is a top view showing a fusion cage according to one embodiment of the present fusion cage design in an open or expanded position;

FIG. 2 is a top view showing the fusion cage of FIG. 1 in a closed or collapsed position;

FIG. 3 is a right side view of the fusion cage of FIG. 1 in a collapsed or inserted position and showing serrations on the top and bottom of the cage;

FIG. 4 is an overhead view of the insertion instrument and open positioned fusion cage assembly of another design with the fusion cage being captured and held by the insertion instrument;

FIG. 5 is a side view of the captured fusion cage assembly and insertion instrument of FIG. 4;

FIG. 6 is a perspective view of the captured fusion cage assembly in and open and rotated position, but still captured by the insertion instrument;

FIG. 7 is a perspective view of the fusion cage assembly in a closed (inserted) position;

FIG. 8 is a side, elevation view of the fusion cage assembly of FIG. 7;

FIG. 9 is a side elevation view of the captured fusion cage assembly and insertion instrument of FIG. 4, as it is inserted adjacent to the spine during practice of the fusion cage assembly insertion method disclosed herein;

FIG. 10 is a section view taken along line 10-10 of FIG. 9, where the insertion instrument is inserted to place the fusion cage assembly adjacent to the spine;

FIG. 11 is the same view as in FIG. 10, but with the insertion instrument withdrawn to an intermediate withdrawal position;

FIG. 12 is the same view as in FIGS. 10 and 11, but with the insertion instrument being advanced further into the body to commence rotation of the fusion cage;

FIG. 13 is the same view as in FIGS. 10-12, but with the insertion instrument being advanced further into the body to complete rotation of the fusion cage into position in the spine;

FIG. 14 is the same view as in FIGS. 10-13, but with the insertion instrument being removed from the body to leave the fusion cage into position in the spine;

FIG. 15 is the same view as in FIGS. 10-14, but with insertion instrument fully removed from the body to leave the spinal fusion cage assembly in position in the spine;

FIG. 16 is a perspective view of the insertion instrument and captured fusion cage assembly as depicted in FIG. 13; and

FIG. 17 is a perspective view of the inserted fusion cage assembly, as depicted in FIG. 15,

The drawings will be described in greater detail below.

### DETAILED DESCRIPTION

The present disclosure is directed to an interbody fusion cage assembly that is used in spinal fusion procedures, such as a transforaminal lumbar spinal fusion procedure, by way of example. More particularly, the present disclosure is directed to an articulated fusion cage assembly that can be adjusted in configuration to facilitate the insertion of the cage assembly between adjacent vertebrae in the spine, such as the lumbar region. The fusion cage assembly of the present disclosure may be inserted by the use of minimally invasive surgical techniques wherein relatively small incisions are made in the patient and instruments are utilized to guide the cage assembly to the desired location between adjacent vertebrae. The articulated nature of the cage assembly allows the cage assembly to be disposed at an angle that facilitates the insertion of the cage assembly around the neural elements and reduces the displacement or impact on the nerve roots during the insertion process.

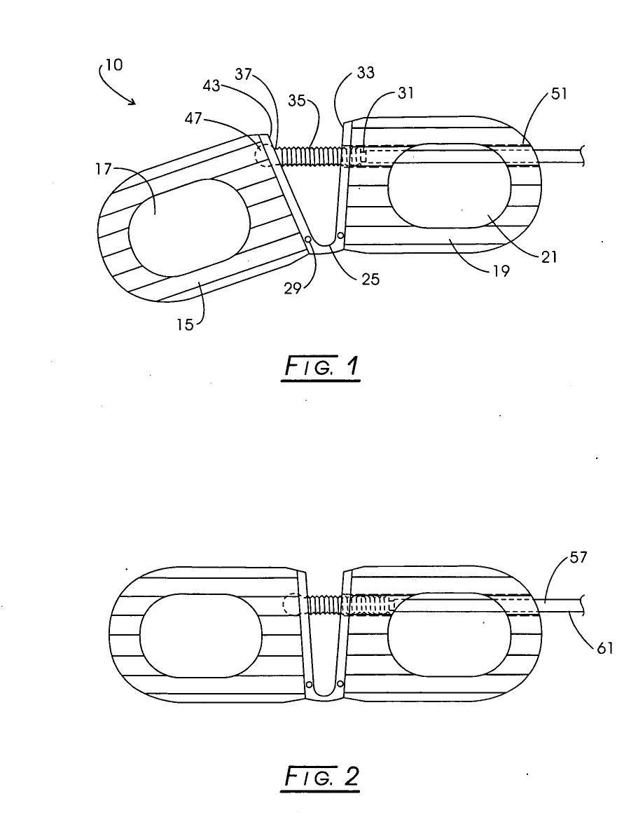

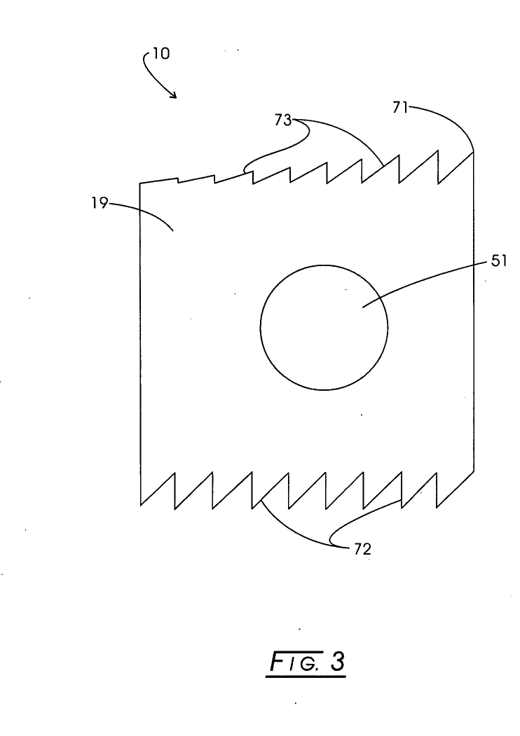

Referring now to the embodiment depicted in FIGS. 1-3, and to FIGS. 1-2 in particular, a fusion cage assembly, 10, has a first spacer member or chamber, 15, and a second spacer member or chamber, 19, that are connected together by an articulating mechanism, such as a hinge, 25, to form the complete fusion cage assembly. The cage assembly may be made of reinforced carbon fiber, PEEK (poly ether ether ketone) polymer material, titanium, or other suitable biomaterial. Hinge 25 may be made of nitinol, titanium, or other biocompatible material. Hinge 25 may incorporate holes, 29, in the hinge material to assist in connecting to the cage assembly material. Holes 29 provide an opening in which the bone or bone replacement material can protrude to form a secure bond between the cage assembly and the hinge.

Alternatively, hinge 25 could be created by using a mechanism similar to one seen in a door hinge, wherein one chamber of the fusion cage assembly pivots in relation to the other. Chambers 15, 19 of fusion cage assembly 10 interdigitate at hinge 25, allowing them to pivot in relation to each other. It will be appreciated that other types of hinge or articulating mechanisms known to those of ordinary skill in the art are possible as well without departing from the spirit and scope of the present disclosure.

In one embodiment, first space member 15 and second spacer member 19 may be generally elliptical in shape when looked at from above; and openings 17 and 21 may be provided in first and second spacer members, respectively, as shown in FIGS. 1 and 2. In one embodiment, fusion cage assembly 10 may have a “lordotic” shape, wherein the front of cage assembly 10 is taller than the back. Cage assembly 10 may have serrations, 73, provided on the top and bottom of the cage assembly. First and second spacer members 15, 19 of cage assembly 10 may be the same length or may vary in length and size with, for example, second spacer member 19 being longer and making up to 70% of the total length of cage assembly 10. First and second spacer members 15, 19 may be designed to fit between and properly space adjacent vertebrae in the lumbar region of the spine. Fusion cage assembly 10 may be used when a disk is removed from between the vertebrae and it is necessary to use cage assembly 10 to provide the necessary spacing between the vertebrae and to stabilize the vertebrae after the disc has been removed. In most applications, bone or bone graft substitute will be positioned in openings 17, 21 of first and second spacer members 15, 19, so that the bone can fuse with the adjacent vertebrae to complete the repair on the spine.

In one embodiment, a threaded passageway, 31, extends from opening 21 in second spacer member 19 to an end, 33, of second spacer member 19 that is adjacent to first spacer member 15. Threaded passageway 31 may be metallic and made of material such as, for example, titanium. Passageway 31 may be encased within the wall of trailing chamber 19. An advancement mechanism, such as a threaded rod/screw, 35, may be positioned in threaded passageway 31, so that the threads on the rod engage the threads on threaded passageway 31. An end, 37, of rod 35 that is spaced apart from opening 21 in second spacer member 19 is disposed to engage an edge, 43, of first spacer member 15. A pivoting foot or ball in a socket, 47, design may be employed on the end of threaded rod 35 that engages edge 43 of first spacer member 15. The pivoting foot or ball and socket design facilitates angulation of the cage assembly as the hinge is deployed. A port, 51, may extend through a portion of second spacer member 19 that is on the opposite side of opening 21 from threaded passageway 31. Port 51 may extend into opening 21 and is disposed to be in alignment with threaded passageway 31. Port 51 may be threaded to facilitate placement of a cage assembly inserter or tool, 57, having a shaft, 61, as shown in FIG. 2, which can be inserted into port 51 and advanced to engage threaded rod 35 so that tool 57 can used to rotate and advance threaded rod 35. Port 51 may be placed as far anteriorly (in the front) as possible so that inserted tool device 57 occupies the least amount of space within chamber 21.

In use, fusion cage assembly 10 of the present disclosure is in the collapsed position shown in FIG. 2 with end 33 of second spacer member 19 positioned immediately adjacent edge 43 of first spacer member 15 when the cage assembly is initially beginning to be inserted into the patient. Fusion cage assembly 10 in this collapsed positioned is advanced into an incision made in the patient to position fusion cage assembly 10 between adjacent vertebrae in the spine, such as in a transforaminal lumbar spinal fusion procedure. As fusion cage assembly 10 is inserted, it must move around the neural elements that are positioned adjacent the area where fusion cage assembly 10 will be located between the adjacent vertebrae. Essentially, fusion cage assembly 10 must be inserted and rotated around the neural elements to position the fusion cage assembly in the desired location.

To reduce the intrusion of fusion cage assembly 10 into the body of the patient and to reduce the amount of displacement that may be necessary for the spinal cord it is desirable to articulate or bend the fusion cage assembly so that it will more easily move around the spinal column. This becomes especially important when fusion cage assembly 10 is inserted through relatively small incisions utilizing an access tube or cannula. In such situations, there is little room for maneuverability, and a straight position of the cage assembly during the initial insertion process is desirable. When fusion cage assembly 10 is inserted into the body so that first spacer member 15 is extending past the dural sac, tool 57 can be turned, much like a screwdriver, to advance threaded rod 35 in threaded passageway 31. Pivoting foot or ball in socket 47 on the end of threaded rod 35 permits edge 43 of first spacer member 15 to be advanced away from end 33 of second spacer member 19 as threaded rod 35 is advanced via operation of tool 57. The advancement of threaded rod 35 causes first spacer member 15 to pivot away from second spacer member 19 around pivot point or hinge 25 that connects first spacer member 15 to second spacer member 19. Threaded rod 35 is advanced until first spacer member 15 is in the desired angular relationship with respect to second spacer member 19 and fusion cage assembly 10 can be advanced into the patient in a direction that is less intrusive and not injurious to the body of the patient. Tool 57 can be used to adjust the angular position between first spacer member 15 and second spacer member 19 to facilitate the insertion of fusion cage assembly 10. As first spacer member 15 is advanced between the adjacent vertebrae and around the spine, threaded rod 35 can be advanced to increase the angle between first spacer member 15 and second spacer member 19. Increasing the angle allows fusion cage assembly 10 to progressively move to the angulated position so as to allow fusion cage assembly 10 to be positioned into the proper location between the adjacent vertebrae.

When fusion cage assembly 10 is fully inserted between the adjacent vertebrae, threaded rod 35 will have been advanced so that fusion cage assembly 10 is in the angulated position shown in FIG. 1. When fusion cage assembly 10 has been angulated, tool 57 can be disengaged from threaded rod 35 and retracted until the threads in tool 57 are engaged with threads within port 51. The cage assembly is then further advanced by using an impactor and properly located between the adjacent vertebrae. Tool 57 then is removed from second spacer member 19. The end of tool 57 that engages threaded rod 35 will have a mechanism, as is well known in the art, to engage the threaded rod so that the tool can cause the threaded rod to be rotated in threaded passageway 31. It will be appreciated that other advancement mechanisms for opening and collapsing first and second spacer members 15 and 19, and other types of tools for selectively advancing the advancement mechanism are possible as well without departing from the spirit and scope of the present disclosure. Further details of this new fusion cage assembly method will be detailed below in connection with another embodiment of the disclosed fusion cage assembly.

If desired, a shoulder (not shown) can be positioned in threaded passageway 31 adjacent opening 21 to act as a stop for threaded rod 35. The shoulder will prevent threaded rod 35 from being advanced into opening 21 in second spacer member 19.

First and second spacer members 15, 19 of cage assembly 10 could be symmetric or asymmetric in size. Leading chamber 15 could be smaller (with 40:60 ratio with trailing chamber 19. Such a configuration would decrease stresses on leading chamber 15 as the tallest portion of cage assembly 10 would be located on trailing chamber 19. This would, in turn, decrease the risk of shearing and stripping of advancing mechanism 35.

If desired, hinge 25 could be created with a scored metal rod. Hinge 25 is contained between chambers 15, 19, and the wings of the scored metallic rod are initially deployed to keep cage assembly 10 in a collapsed position. As cage assembly 10 is partially inserted, the wings of the scored metallic rod could be retracted allowing the rod to elongate between chambers 15, 19, which would angulate the cage assembly.

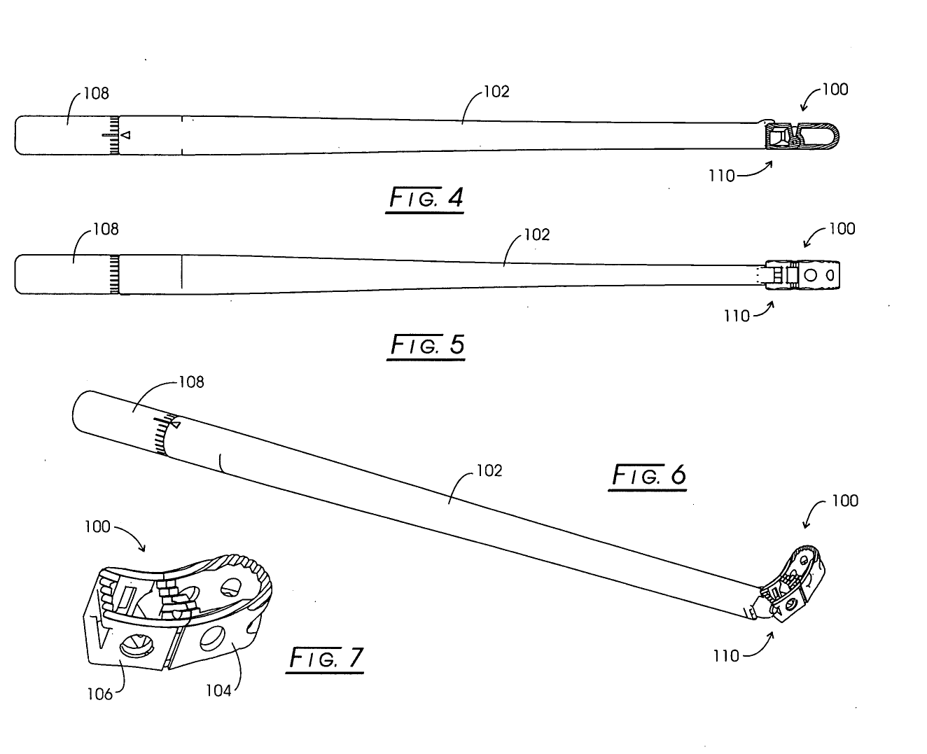

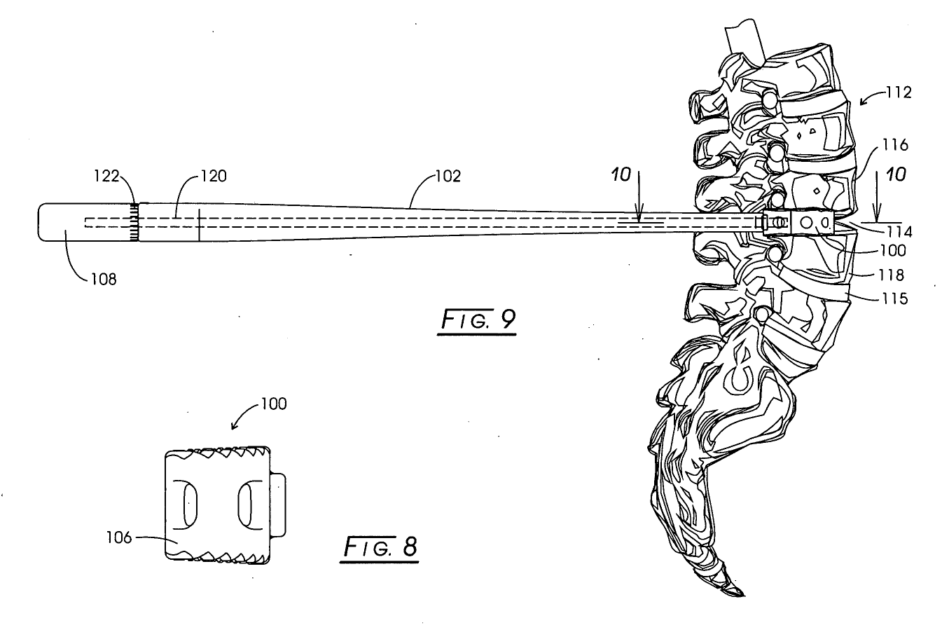

Referring now to another embodiment of the disclosed fusion cage assembly, reference is made to FIGS. 4-17, where a fusion cage assembly, 100 (see FIGS. 7 and 8), is seen captured by an insertion instrument, 102 (FIGS. 4-6). Like the embodiment depicted in FIGS. 1-3, fusion cage assembly 100 is composed of two spacer members or elements, a leading spacer member, 104, and a trailing spacer member, 106. The top and bottom surfaces of both spacer members are serrated in order to assist the adjacent vertebrae in retaining the inserted fusion cage assembly in position. Again like before, insertion instrument 102 captures fusion cage assembly 100 for its insertion into position where a disk has been removed from the spine. Like the previously described embodiment, fusion cage assembly is articulated about a pivot point. Insertion instrument 102 is elongate having a handle region, 108, and a capture region, 110. Its operation will be described in greater detail below in connection with the fusion cage assembly procedure.

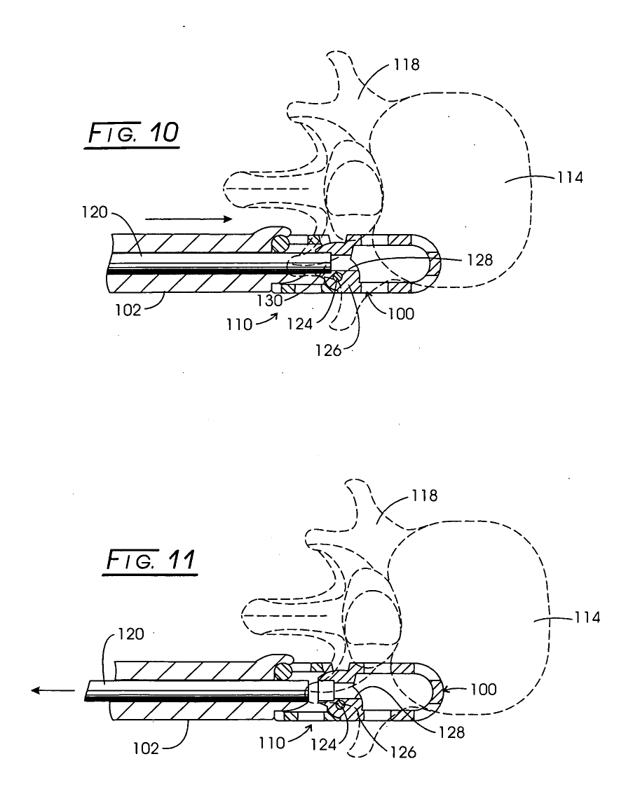

Referring now to FIG. 9, insertion instrument 102 and captured fusion cage assembly 100 have been placed in position adjacent to a spine, 112, and adjacent to a space, 114, created by the prior removal of a disk (similar to a disk, 115) located between an upper vertebra, 116, and a lower vertebra, 118. Shown in phantom disposed in the interior of annular instrument handle 102 is a rod, 120, that actually captures fusion cage assemble 100. Handle 108 contains a dial, 122, that provides a readout to the surgeon indicating the distance rod 120 has been extended or retracted with respect to instrument 102.

In particular, spacer members 104 and 106 are held together by a pivot pin, 124, which is located in a body, 126, having a forward surface, 128. The distal end, 130, of rod 120 bears against surface 128 of body 126 and, thus, keeps fusion cage assembly 100 in an open position, as depicted in FIG. 10. When the surgeon rotates handle region 108 a defined amount as determined by dial 122, rod 120 withdraws towards the proximal end of insertion instrument 102 and, thus, no longer is in contact with surface 128. This partially withdrawn position of rod 120 now permits leading spacer member 104 to rotate about pin 124 to form an arcuately shaped fusion cage assembly and also rotate about another pin, 132, with respect to trailing spacer member 106, because spacer member 106 still is held by road 120.

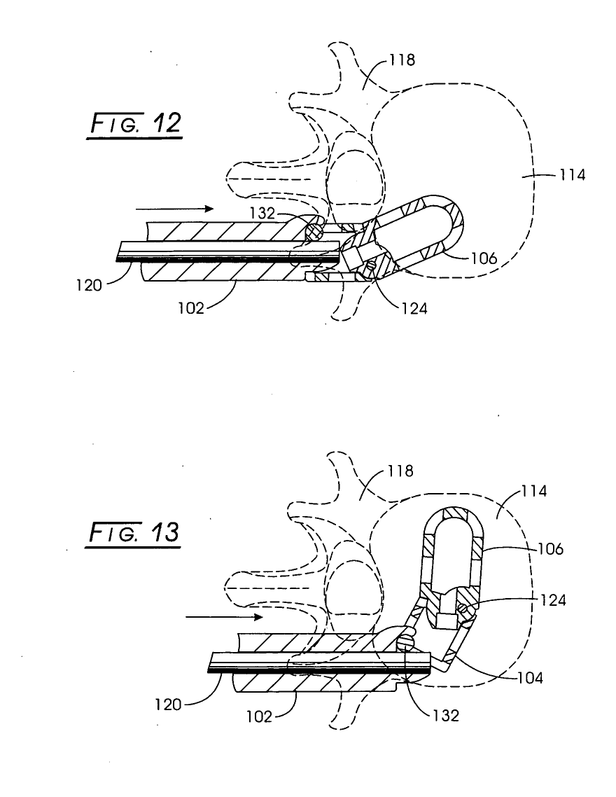

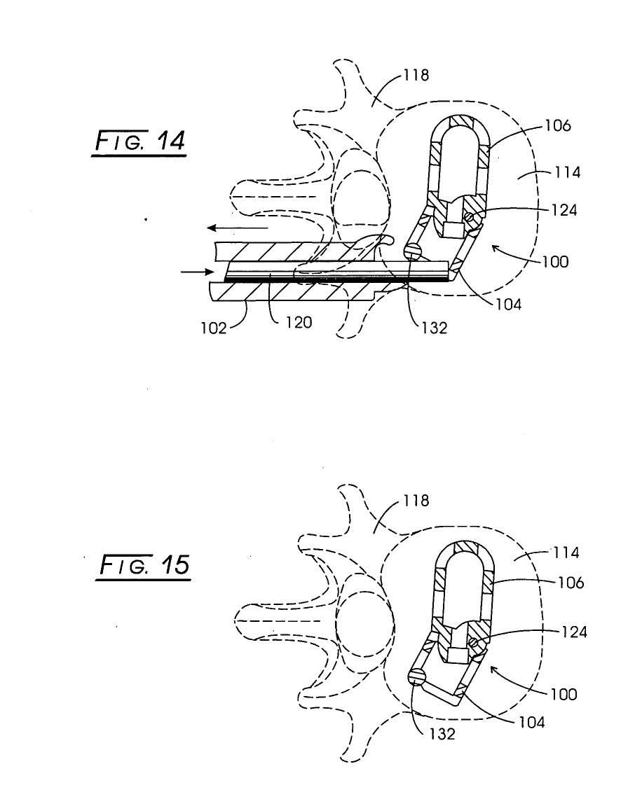

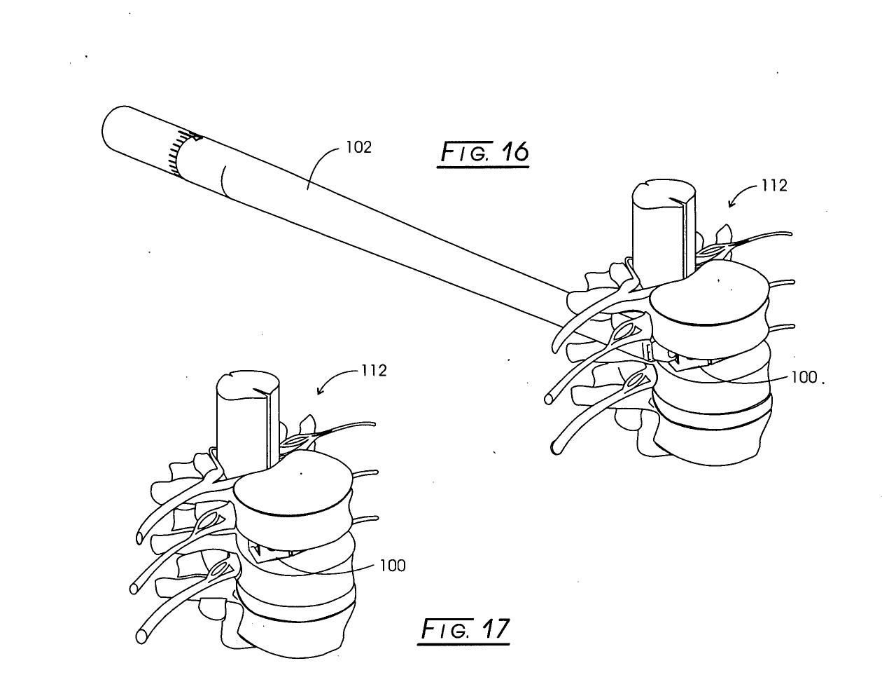

At this juncture of the insertion procedure and as depicted in FIGS. 12 and 13 (and associated FIG. 16), the surgeon commences to again push insertion instrument further into the patient. Leading spacer member 104, however, encounters tissue that causes it to rotate about pins 124 and 132 into space 114. Such rotational movement prevents leading spacer member 104 from contacting the spinal cord or any nerves and other sensitive tissue associated with spine 112. The arcuate or closed position of fusion cage 100 is maintained when insertion instrument 102 is withdrawn by the surgeon and out of contact with fusion cage assembly 100, as depicted in FIGS. 14 and 15. At this juncture, the procedure is complete and insertion instrument 102 is totally removed from the patient, as seen in FIG. 17. While the spinal cord is shown moved aside from the path of the advancing fusion cage assembly, the retractor that is used therefor has been omitted for ease in illustrating the disclosed spinal cage assembly.

While the device, system, and insertion method has been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

We claim:

1. A spinal cage system for inserting a spinal cage assembly into a spine to separate and support adjacent spinal vertebrae, which comprises:
(I) a spinal cage assembly comprising:
(a) a leading cage member;
(b) a trailing cage member; and
(c) an articulating mechanism adapted to connect said leading cage member to trailing second cage member and to permit said leading and trailing cage members to move relative to each other; and
(II) an insertion instrument adapted to capture said spinal cage assembly for insertion of said spinal cage assembly into a spine and to rotate said leading and trailing members relative to each other to achieve a desired orientation in said spine between adjacent vertebrae.

2. The spinal cage assembly of claim 1, wherein said articulating mechanism comprises a pin.

3. The spinal cage assembly of claim 1, wherein said leading cage member and said trailing cage member both are formed from a frame having a substantially hollow interior.

4. The spinal cage assembly of claim 1, wherein said insertion instrument is annular and has a movable rod disposed therewithin capable of capturing said spinal cage assembly for insertion into a patient.

5. A spinal cage assembly to separate and support adjacent vertebrae in a spine, comprising:
(a) a first spacer member for insertion between the adjacent vertebrae;
(b) a second spacer member for insertion between the adjacent vertebrae;
(c) an articulating mechanism located between said first and second spacers to connect the first spacer member to the second spacer member so that the first and second spacer members move relative to each other; and
(d) an advancing mechanism located between the first and second spacer members, the advancing mechanism being disposed to move the first and second spacer members relative to each other around the articulating mechanism wherein the angle of the first spacer member relative to the second spacer member facilitates the insertion of the cage around the spinal cord, the advancing mechanism being operable to position the first and second spacer members in a desired orientation relative to one another when the cage is fully positioned between the two adjacent vertebrae.

6. The cage of claim 5, wherein the articulating mechanism comprises a hinge.

7. The cage of claim 5, wherein the advancing mechanism comprises a rod that engages the cage.

8. A spinal fusion cage assembly adapted to separate and support adjacent vertebrae in a spine, comprising:
(a) first and second spacer members mounted for articulation relative to each other and being configured for insertion between adjacent vertebrae; and
(b) an advancing mechanism located between the first and second spacer members, the advancing mechanism being operable to articulate the first and second spacer members relative to each other for insertion between the adjacent vertebrae.

9. A spinal fusion cage assembly adapted to separate and support adjacent vertebrae in a spine, comprising:
(a) a leading spacer member and a trailing spacer member mounted together with a first pin for articulation relative to each other and being configured for insertion between adjacent vertebrae, said trailing spacer member fitted with a second pin; and
(b) an advancing mechanism having a proximal end and a distal end, the distal end designed to capture said trailing spacer member to insert said spinal fusion cage assembly into a patient between adjacent spinal vertebrae, wherein said spinal fusion cage assembly rotates about said second pin relative to said advancing mechanism distal end and said leading spacer member rotates about said first pin relative to said trailing spacer member for placement of said spinal fusion cage assembly between said adjacent vertebrae.

10. The spinal fusion cage assembly of claim 9, wherein each spacer member has a top surface and a bottom surface, one or more of said spacer member top and bottom surface being serrated.

11. The spinal fusion cage assembly of claim 9, wherein one or more of said spacer members are formed from a hollow frame assembly.

12. The spinal fusion cage assembly of claim 9, wherein said advancing mechanism comprises an elongate annulus having a handled proximal end and a rod disposed within said annulus that has a proximal end connected to said handle and a distal end that captures said second pin.
